(19) United States
(12) Patent Application Publication
(10) Pub. No.: US 2025/0011707 A1
(43) Pub. Date: Jan. 9, 2025

(54) Device and Method for Separation, Concentration, and Collection of Algal Biomass from Aqueous or Marine Culture

(71) Applicant: Porelogix Ultrafiltration Systems, LLC, Omaha, NE (US)

(72) Inventor: Conor Campbell, Omaha, NE (US)

(73) Assignee: Porelogix Ultrafiltration Systems, LLC, Omaha, NE (US)

(21) Appl. No.: 18/707,653

(22) PCT Filed: Nov. 22, 2022
(86) PCT No.: PCT/US2022/050765

§ 371 Date: May 6, 2024

Related U.S. Application Data
(60) Provisional application No. 63/264,395, filed on Nov. 22, 2021.

(51) Int. Cl. C12M 1/00; C12M 1/26; C12N 1/12

(52) U.S. Cl. CPC C12M 47/02; C12M 33/14; C12N 1/12

(57) ABSTRACT

Devices and methods for separating, concentrating, and collecting algal biomass are provided. In particular, the devices and methods of the present application may be suitable for collecting algal biomass from aqueous or marine culture. The device includes a filter membrane, a collection housing assembly for collecting algal biomass, and a blower for blowing algal biomass from the collection housing assembly.

### CROSS-REFERENCES TO RELATED APPLICATIONS

This Non-Provisional Patent application claims the benefit of and priority to PCT Application Serial No. PCT/US2022/050765, filed Nov. 22, 2022, entitled “Device and Method for Separation, Concentration, and Collection of Algal Biomass from Aqueous or Marine Culture,” which claims the benefit of and priority to U.S. Provisional Patent Application Ser. No. 63/264,395, filed Nov. 22, 2021, the entire contents of both applications of which are hereby incorporated herein by reference.

### TECHNICAL FIELD

Devices and methods for separating, concentrating, and collecting algal biomass are provided. In particular, the devices and methods of the present application may be suitable for collecting algal biomass from aqueous or marine culture.

### BACKGROUND

Algal biomass is used for a variety of products including petroleum derivatives such as biofuels, industrial lubricants, green chemicals, bioplastics, and cosmetics. Algae (algal biomass) is also used for food products such as supplements, food additives, food thickeners, and biopharmaceuticals. However, harvesting algae has proved to be difficult on a large scale. Large-scale commercial algal biomass farms lack an economical and efficient method to separate, concentrate, and collect algal biomass from an aqueous or marine culture. Current methods do not yield an effective amount of biomass at industrial scale and require high energy and capital costs.

### SUMMARY

The device and methods described in embodiments relate to the separation, concentration, and collection of algal biomass from an aqueous or marine culture for the purpose of creating high-value products and carbon sequestration.

Algal biomass harvesting is a challenge because of the small size of algal cells (3-30 μm diameter), their similar density to water, and the large water volumes that must be handled to recover the biomass. In some instances, collection of 10 kg of algal biomass from a 3 g/L algae suspension takes 3,300 L of water. Recovery of the biomass from the aqueous or marine culture medium may contribute between 20 and 30% of the total cost of producing the algal biomass.

Algae harvesting includes one or more solid-liquid separation steps, including the concentration and collection processes. The most frequently used concentration technologies are coagulation, flocculation, flotation, centrifugation, filtration (screen, ultrafiltration (UF) membrane), and gravity sedimentation. UF membrane technology according to embodiments described herein can remove bacteria from used algal culture media, which impacts algae growth, while retaining residual nutrients (total dissolved solids (TDS)). The UF membrane technology described herein allows expensive algal culture media to be recycled.

Current UF algal biomass harvesting systems have difficulties with irreparable fouling of the UF membranes, low concentration rates that do not yield an effective amount of biomass at industrial scale, high energy costs, and high capital costs. The current devices, methods, and systems separate, concentrate, and collect the algal biomass via UF in a manner that minimizes the irreparable fouling of the UF membranes and achieves high concentrations of the algal biomass at low energy utilization.

The device and method utilize hydrophilic modified polyacrylonitrile (PAN) hollow fiber membranes in a dead-end configuration, to continuously or batch feed algal-containing liquid. The device described herein incorporates a UF membrane regeneration process that includes air scour-forward flush and air scour-back flush. During the separation process, the algal biomass is concentrated in the collection housing assemblies and harvested via a harvesting line with advanced air displacement technology that rapidly collects the concentrated algal species.

The device is designed with flexibility by isolating membranes for variable sized harvests to maintain a high concentration of algal product. The device incorporates multiple valve placements on the feed, reject, collection, and air lines.

The device and method are controlled via sensors, a control panel, user interface, and computer processors specifically designed to gather valuable data relating to the algal feed product including concentration, amount, membrane performance, and amount of algal biomass collected. The device and method control the process steps of separation and collection.

### BRIEF DESCRIPTION OF THE DRAWINGS

FIG. 1 is a back perspective view of the device according to an embodiment of the disclosure;

FIG. 2 is a back view of the device according to an embodiment of the disclosure;

FIG. 3 is a front perspective view of the device according to an embodiment of the disclosure;

FIG. 4 is a back view of the device according to an embodiment of the disclosure;

FIG. 5 is a right-side view of the device and control panel according to an embodiment of the disclosure;

FIG. 6 is a front perspective view of the device according to an embodiment of the disclosure;

FIG. 7 is a top view of the device according to an embodiment of the disclosure;

FIG. 8 is a left-side view of the device according to an embodiment of the disclosure;

FIG. 9 is a perspective view of collection housing assemblies according to an embodiment of the disclosure;

FIG. 10A is a perspective view of an air vent assembly in accordance with an embodiment of the disclosure;

FIG. 10B is a perspective view of a flexible valve in accordance with an embodiment of the disclosure;

FIGS. 11A and 11B are front and back views of a collection housing assembly according to an embodiment of the disclosure;

FIG. 12 is a diagram of membrane configurations according to an embodiment of the disclosure; and

FIG. 13 is a flow diagram of the method of concentration and collection of biomass according to an embodiment of the disclosure.

### DETAILED DESCRIPTION

The device and method are directed to the separation, concentration, and collection of algal biomass from an aqueous or marine culture for the purpose of creating high-value products. The device and method provide a low fouling, low energy, cost effective scalable ultrafiltration system, which is capable of operating without rapid deliberating, irreversible scaling, and fouling of the membranes. The device and method concentrate algal biomass using a continuous or batch feed for further processing. The device and method recycle 95-98% of the permeate water without rejecting the total dissolved solids (TDS). The design of the device with a flexible number of valves and membranes increases algal yield and minimizes waste.

The device and methods allow for algal biomass, collected in a collection housing assembly, to be collected by inducing airflow in the collection housing assembly without causing airlock. The device footprint is smaller than current technology and can be operated more efficiently and at a lower cost. Additionally, no booster pump is needed. The sensors, control panel, and processors allow for ease of use and allow users to make data-driven decisions.

The device also incorporates a prefilter, feed pump, back flush pump, air blower, programmable logic controllers (PLC), control panel (and all components within), software, feed pumps, automatic valves, check valves, manifolds, flow meters, pressure meters, low-pressure air valves and other components working in conjunction to separate, concentrate, and collect algal biomass from an aqueous or marine culture in the method described below. Algal biomass may include a small amount of liquid but is more concentrated than the influent liquid. The concentration provided by the device depends on the type of algae species and starting concentration of the influent liquid (typically around 0.02 MG/L). Additionally, the time to filtration and size of the batch feed also affects the concentration. In embodiments, 1000 gallons of influent liquid are needed per membrane with a concentration target of 0.2 MG/L. However, some embodiments have demonstrated the harvested algae is 40 times more concentrated than the influent water. The device dewaters the influent liquid 98-99% thus reducing the size of the centrifuge needed by 98-99% for algae harvested with device 10.

The following terms will be used throughout this specification and will have the following definitions unless otherwise indicated.
“Permeate” The treated product stream. Water that has passed through UF membrane.
“Reject” The product being sent to drain “waste.”
“Regeneration” The process(es) or sequences of processes of regenerating the membranes by removing the concentrated solids (algae) and other oils from the pressurized membrane to maintain optimal efficiency.
“Dead-End” The filtration method where there is no continuous waste flow. The membrane is periodically flushed to remove retained solids from the membrane surface.
“Cross-Flow” The filtration method where a portion of the feed water is continuously recirculated and/or flows to waste to prevent retained solids from building up on the membrane's surface.
“Tangential flow” A flow along the tangent or across the surface of the membrane.
“VFD” (Variable Frequency Drive) Device that allows for specific control of a pump's flow rate.
“Hydrophilic” Having a strong affinity to water.
“Flux” The amount of permeate per square inch of membrane surface per day.
“Continuous feed” The process in which the feed source is continuously fed into the system without a predetermined aqueous feed amount.
“Batch feed” The process in which the aqueous feed source is defined by how much is available or predetermined by the amount of concentrated product desired.
“Outside-In Flow” The flow direction of the aqueous feed source from the outside of the fiber which passes through to the inside of the fiber where it is collected as treated (permeate) water, thus trapping the algae on the outside of the fiber and in the collection housing.
“Inside-Out Flow” The flow of the aqueous feed source traveling down the bore (inside) of the fiber passes through the fiber to the outside where it is collected as treated (filtrate) water.
“Algae/Algal Species” A large and diverse group of photosynthetic eukaryotic organisms. Specifically relating to a polyphyletic grouping that includes species from multiple distinct clades. This includes organisms ranging from unicellular microalgae, such as Chlorella, Spirulina (known as cyanobacterium), Nannochloropsis (including but not limited to Oceanica), Scenedesmus, Prototheca and the diatoms, to multicellular forms such as seaweed. In addition, Charophyta, a division of green algae which includes, for example, Spirogyra and stonewarts is also considered algae. No definition of algae is generally accepted. One definition is that algae “have chlorophyll as their primary photosynthetic pigment and lack a sterile covering of cells around their reproductive cells.”
“TDS” Total Dissolved Solids.
“NTIPS” Nonsolvent Thermally Induced Phase Separation is a procedure for membrane preparation.
“TIPS” Thermally Induced Phase Separation is a procedure for membrane preparation.
“NIPS” Nonsolvent-Induced Phase Separation is a procedure for membrane preparation.

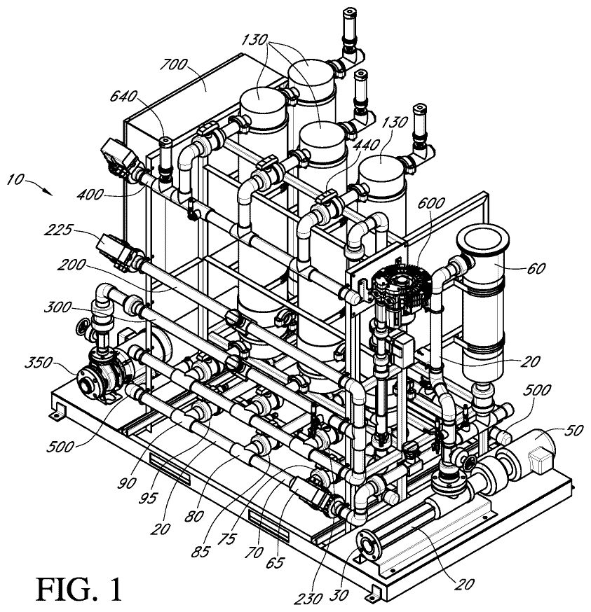

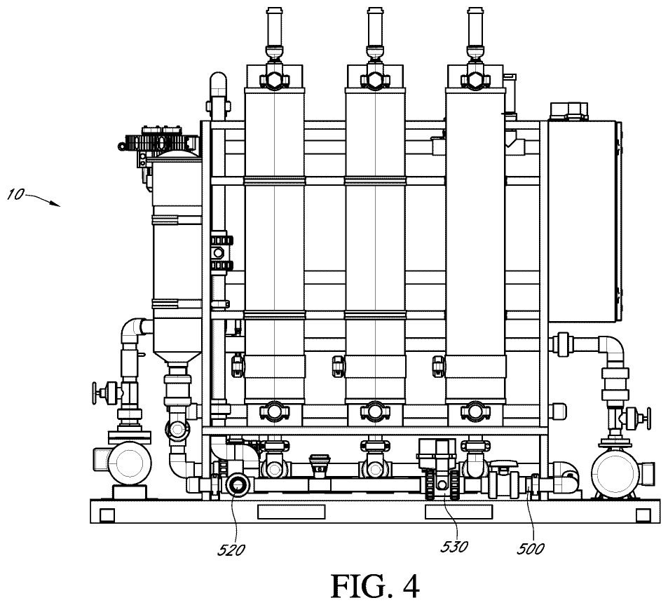

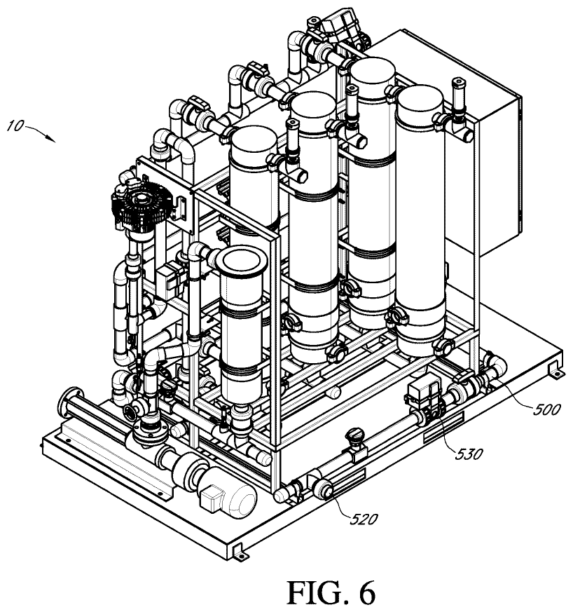

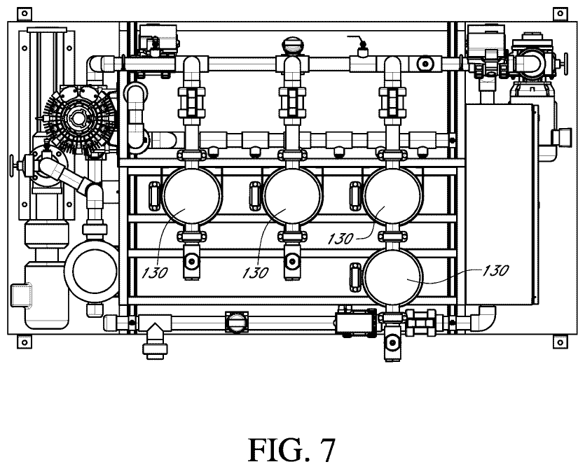

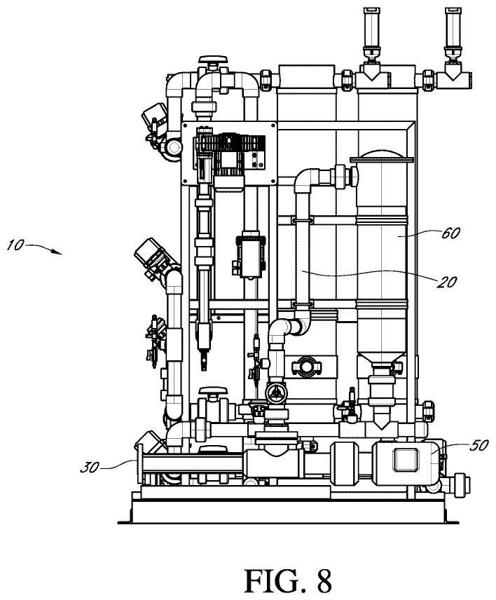

A collection and separation device 10 according to embodiments is described with reference to FIGS. 1-8. The device assembly is configured for use in skid-based or large-scale ultrafiltration plants.

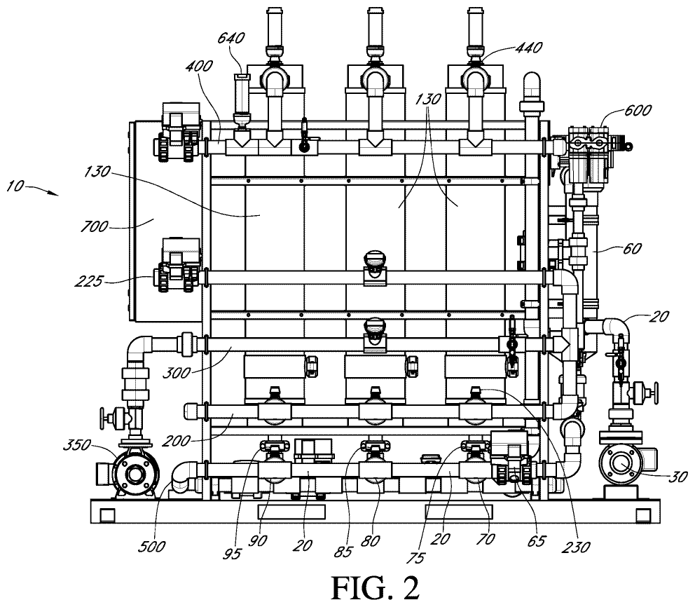

Device 10 is a system of multiple lines and membranes for the separation and concentration of solids, such as algal biomass, from a liquid, such as an aqueous or marine culture. The multiple lines and construction of the device minimize the irreparable fouling of the UF membranes and concentrate the algal product in pressurized collection housings at low energy in an outside-in, dead-end configuration, with continuous or batch feed. Device 10 may undergo membrane regeneration with air scour-forward flush/air scour-back flush. With reference to FIGS. 1 and 2, device 10 comprises feed line 20, permeate line 200, backflush line 300, drain line 400, harvest line 500, blower 600, and control panel 700. The lines may be made of pipe material for liquid to flow therethrough. When the device is assembled, the lines are connected to separate and concentrate solids, such as algae, from the liquid.

In FIGS. 1, 2, and 8, influent liquid, such as aqueous or marine culture, from a pond or holding tank (not shown) enters device 10 via influent liquid inlet 30. A hose or other liquid connection may be connected to influent liquid inlet 30. Feed pump 50 moves influent liquid from a pond or holding tank at a controlled rate through feed line 20. In embodiments, feed pump 50 is a pressure feed pump for moving a continuous flow of influent liquid. In embodiments, the rate of continuous flow is 0.04 to 50 psi. Influent liquid moves through feed line 20 to prefilter device 60. Prefilter device 60 removes large solids, such as gravel, sand, and tree material from the influent liquid. Prefilter device 60 may remove large solids from influent liquid through gravity filtration, flocculation, air floatation, or any other removal method. Prefilter device 60 may include a prefilter flow meter for measuring flow into prefilter device 60, prefilter control and check valves for controlling flow into and out of the prefilter device, prefilter sensors, such as a spectrophotometer for measuring properties of influent liquid, and prefilter transmitters for transmitting information about the prefilter device and influent liquid. In some embodiments, device 10 may not include a prefilter device 60 and prefiltering may be performed outside of device 10.

With reference to FIGS. 1 and 2, feed pump 50 moves influent liquid through feed line 20 to inlet ports 70, 80, and 90. Feed pump 50 may be a variable frequency drive (VFD) device. Each of ports 70, 80, and 90 directs the influent liquid to one or more collection housing assemblies 130 having one or more membrane filters for separating biomass from the influent liquid. Ports 70, 80, and 90 may be opened or closed using corresponding flex valves 75, 85, and 95. Flex valves 75, 85, and 95 may be manually opened or closed or may be opened or closed by commands from control panel 700. Although device 10 is depicted as having a feed line 20 with three ports and three flex valves, any number of ports and flex valves may be utilized.

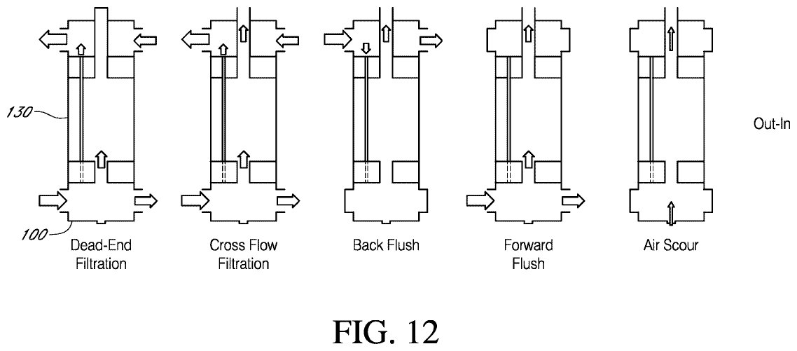

In some embodiments, only one port is opened for inflow of influent liquid. For example, if the flow of influent liquid is low, one opened port, collection housing assembly 130, and one membrane may be sufficient to separate biomass from the influent liquid. Collection housing assembly 130 houses a membrane 100 in a dead-end configuration as shown in FIG. 12. Referring again to FIGS. 1 and 2, if the flow of influent liquid is high, multiple or all of the inlet ports 70, 80, and 90 may be opened for the flow of influent liquid to multiple collection housing assemblies 130 and membranes for separation of biomass from the influent liquid. In an embodiment, influent liquid flows through open ports into collection housing assembly 130 in a direction that is substantially perpendicular to a filter membrane in a dead-end configuration. One or more ports 70, 80, or 90, when in an open position, allow influent liquid to flow or diffuse through one or more membranes to collect biomass.

One or more filter membranes are maintained in collection housing assembly 130. The biomass solids or semi-solids are removed as influent liquid flows through the filter membrane. The permeate (water after flowing through a membrane) is removed from collection housing assembly 130 via permeate line 200.

The feed pressure for device 10 utilizing feed pump 50 is based on the concentration, time restrictions, membrane area, and type of algae species in the aqueous or marine culture. Feed pump 50 feeds influent liquid, such as aqueous or marine culture, to collection housing assembly 130 with drain valve 440 closed and permeate valve 230 open, thus creating dead-end filtration for the algal biomass to be concentrated in collection housing assembly 130. The pore size determines selectivity, allowing the permeate to pass through the hollow fiber membrane.

Device 10 may be designed with one to an infinite number of polyacrylonitrile (PAN) hollow-fiber membranes in an outside-in, dead-end configuration, with a continuous or batch aqueous feed. The number of collection housing assemblies 130 containing the hollow fiber membranes is determined by the amount of aqueous feed needed to be processed, the concentrate rate target, and time. Membranes 100 may be one or more types of (PAN) polyacrylonitrile ultrafiltration membranes prepared via NIPS, NTIPS, or TIPS with variable pore structures, hydrophilicity, and support materials in different lumen sizes or lengths to create the total active surface area per collection housing assembly 130. Any number of membranes may be housed in collection housing assembly 130. Membranes may have asymmetric or symmetric pores ranging in size from 0.005-0.5 microns. Additional chemical solutions may be added to membranes and collection housing assembly 130 to increase hydrophilicity and reduce fouling of membranes.

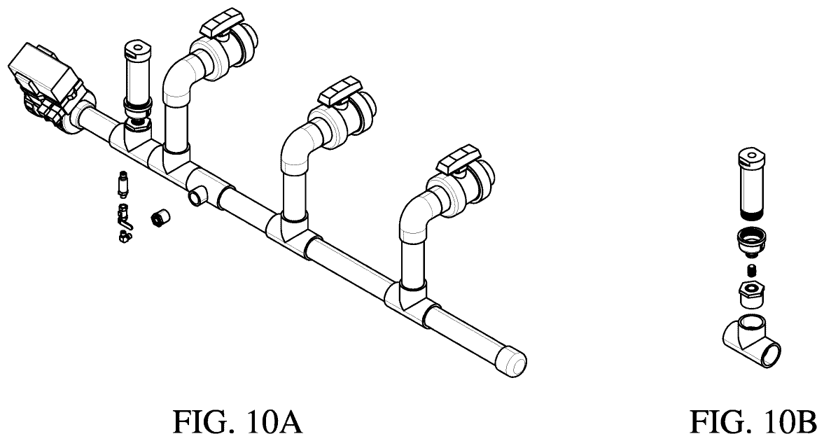

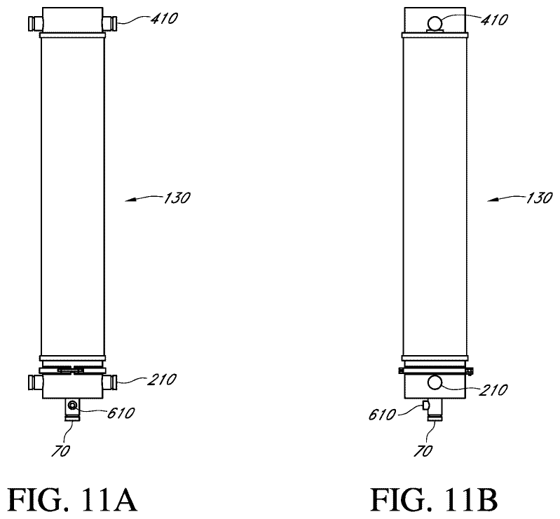

With reference to FIGS. 11A and 11B, collection housing assembly 130 containing the membranes may be pressurized. Each collection housing assembly 130 may have a number of connected ports (inlets and outlets) including feed line and harvest port 70, permeate and backflush port 210, air inlet 610 and drain port 410. Each port may be connected by a flexible design valve as shown in FIGS. 10A and 10B to assist with concentration and collection of algal biomass. In embodiments, referring to FIGS. 1 and 2, feed line 20 and harvest line 500 share the same port 70 for collection housing assembly 130. In embodiments, permeate line 200 and backflush line 300 share the same port 210. In embodiments where ports are shared, some of the lines may also be shared. For example, a portion of feed line 20 may be shared with harvest line 500 and a portion of permeate line 200 may be shared with backflush line 300. In this configuration, influent liquid flows in a first direction into collection housing assembly 130 and harvested biomass flows in a second direction out of collection housing assembly 130 via shared port 70. Permeate flows out of collection housing assembly 130 in a first direction and backflush liquid flows into collection housing assembly 130 in a second direction. In other configurations, each of the feed line 20, harvest line 500, permeate line 200 and backflush line 300 may have separate ports and do not share portions of lines.

Referring again to FIGS. 1 and 2, one or more ports 70, 80, and 90, when in an open position, allow influent liquid to flow into collection housing assembly 130 and flow or diffuse through one or more membranes in a dead-end configuration. Influent liquid flows into collection housing assembly 130, through the membrane, and out port 210 to permeate line 200. Substantially all of the influent liquid flows through the filter membrane in a dead-end configuration.

As shown in FIG. 12, the dead-end configuration is unlike crossflow filtration. In crossflow filtration, a portion of the influent liquid flows across a filter membrane and out of the device to prevent fouling of the membrane. In a dead-end configuration, as described in embodiments, substantially all of the influent liquid flows through the filter membrane. Algal biomass is filtered from the influent liquid and does not flow through the membrane in a dead-end configuration. Algal biomass collects in collection housing assembly 130 until influent liquid flow stops and the harvesting process begins. In embodiments, the biomass collects at the bottom of collection housing assembly 130 until it is removed via harvesting line 500 (described below).

With reference to FIGS. 1 and 2, collection housing assembly 130 is a pressurized device containing one or more filter membranes. Collection housing assembly 130 may maintain hundreds to thousands of (PAN) polyacrylonitrile hollow fiber ultrafiltration membranes with a number of ports for connecting feed line 20, permeate line 200, harvesting line 500, air induction lines (not shown), backflush line 300, and drain line 400.

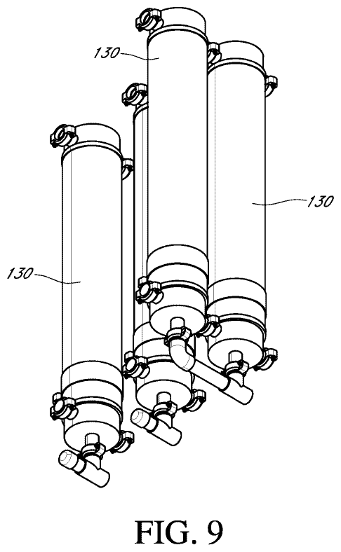

Device 10 may use a single collection housing assembly 130 comprising filter membranes or any number of collection housing assemblies 130 and membranes may be utilized. In the configuration shown in FIGS. 1, 2, 7, and 9, the device has four collection housing assemblies 130. Collection housing assemblies 130 may be connected to feed line 20, permeate line 200, drain line 400, back flush line 300, harvesting line 500, and an air induction line (not shown) with flexible valves as shown in FIGS. 10A and 10B for each port of collection housing assembly 130 to increase algal biomass yield concentration and collection depending on the concentration target, feed concentration, time, and total amount of aqueous or marine culture.

The influent liquid is fed at low pressure from 0.04 to 50 PSI. The operating pressures are determined by the concentration of algae, type of algae species in the influent liquid, and maintaining an appropriate level of flux rate to minimize irreparable fouling of the membranes. The pressure of the collection housing assembly and the rate of feeding influent liquid depends on the total aqueous feed and the number of membranes utilized.

With reference to FIGS. 11A and 11B, permeate (filtered liquid) is removed from collection housing assembly 130 via port 210. In FIGS. 1 and 2, permeate flows through permeate line 200 and out of device 10 via outlet 225. Permeate line 200 may include a flow meter, control valve, pressure transmitters, check valves, flexible port valves, a back flush pump, permeate control and check valves for controlling flow out of collection housing assembly 130, and permeate transmitters for transmitting information about the pressure of permeate line 200 to control panel 700. Permeate leaving device 10 via outlet 225 may be discarded or collected in a collection tank (not shown). Collected permeate may be utilized for the backflush process (described below).

The flow of influent liquid into collection housing assembly 130 and through a membrane is stopped by closing valve 65 either manually or through control panel 700. After the flow of influent liquid is stopped, algal biomass that has been collected in collection housing assembly 130 is removed via harvesting line 500. Air is delivered to collection housing assembly 130 via air induction lines (not shown) connected to an air inlet. Each collection housing assembly 130 has a separate air induction line. In addition to air induction lines, the pressure in each pressurized collection housing assembly 130 may be decreased, creating vacuum pressure. The pressure in the collection housing assembly 130 may be controlled by air blower 600 and air outlet 640. The decrease in pressure, combined with gravity, rapidly collects the concentrated biomass in the collection housing assembly 130 via the harvesting line 500 without causing debilitating airlock. During collection, air is injected from air induction lines into collection housing assembly 130 to force biomass from collection housing assembly 130 into the harvesting line 500 for collection.

Typically, air is not introduced to filtration systems as it can cause airlock and device malfunction. The unique setup of the dead-end configuration membrane, collection housing assembly, feed line, harvesting line, air induction lines, and system valve allows for biomass to be collected from collection housing assembly 130 without causing airlock and for a quick return to use of the system for filtration after collection of biomass from the collection housing assembly.

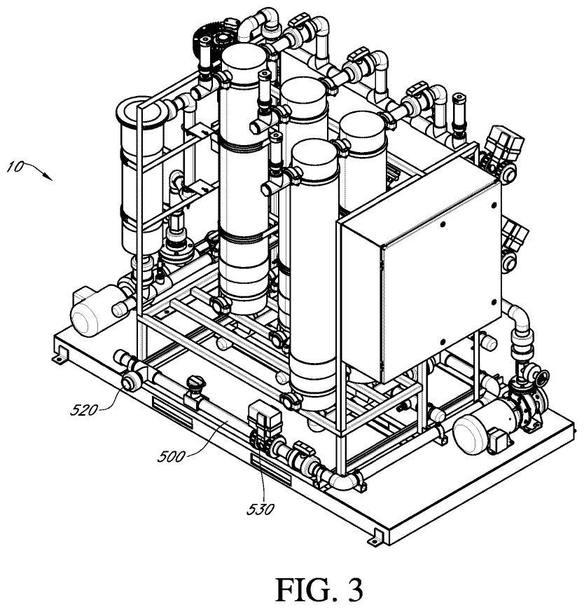

Harvest line 500 is connected to collection housing assembly 130 by port 70. As shown in FIGS. 3, 4, and 6, harvest line 500 of device 10 comprises an outlet 520 and flow totalizer 530. Harvest line 500 may also comprise control valves, pressure transmitters, and a spectrophotometer. Vacuum pressure is created in collection housing assembly 130 to cause algal biomass to be drained out of collection housing assembly 130 through port 70 (FIGS. 11A and 11B) and into harvest line 500. The algal biomass is removed through an air-assisted gravity drain. Harvest line 500 empties the algal biomass from device 10 through harvest outlet 520. The algal biomass emptied from device 10 via harvest line 500 can be utilized for a variety of purposes including biofuels, industrial lubricants, green chemicals, bioplastics, and cosmetics. The algal biomass may also be used for food products such as supplements, food additives, food thickeners, and biopharmaceuticals.

With reference again to FIGS. 1 and 2, after the biomass from collection housing assembly 130 is harvested via harvest line 500, the device 10 may be prepared for continued use or may clean the membranes. If the device 10 is to continue to be used, the device 10 feeds influent liquid via feed line 20 at a high rate of flow, without significant delay, until the membrane is saturated and the collection housing assembly 130 is ready to begin collecting and concentrating algal biomass again. In order to complete this rapid refill process, the permeate port is closed by valve 230, and the drain port is opened by drain valve 440. The feed pump 50 is engaged at a high rate of flow to rapidly refill collection housing assembly 130 with influent liquid. This reduces downtime for device 10.

If after the algal biomass has been removed from collection housing assembly 130, the membrane has solids to be removed. Thus, a membrane regeneration process is performed. The regeneration process comprises an air scour-forward flush process and an air scour-back flush process using blower 600 and air outlet 640 which regenerates the membrane back to its original operational values. The forward flush and back flush processes effectively remove solids that may be retained in the membrane to regenerate the membrane to its original operational values.

During both the back flush and forward flush, air bubbles are injected into the collection housing, dislodging any solids in the hollow fiber (PAN) membrane, thus enabling the forward flush and back flush process to effectively remove the solids from the membrane. In forward flush, the influent liquid is fed through feed line 20 at a flow rate and pressure configured to generate a tangential flow across the membrane to remove solids from the membrane without the transmembrane pressure (TMP) restrictions.

In the back flush process, the filtered liquid (permeate) that has been collected in a tank, such as a back-flow tank, is fed using a black flush pump 250 through back flush line 300 in an inside-out flow path, through the membrane, dislodging algae from the pores regenerating surface of the membrane. Liquid from the forward flush and back flush is removed from collection housing assembly 130 via drain port 410 (FIGS. 11A and 11B) leading to drain line 400. The liquid removed by drain line 400 may be cleaned and returned to a pond if it does not contain cleaning chemicals or environmental toxins. If the liquid removed by drain line 400 contains environmental toxins or impurities, it should be processed for further disposal.

In embodiments, the device is configured into one system as an on-site installation, or as a mobile unit. In another configuration, each unit is designed and configured for easy access on a trailer as a modular unit that can be mobile (transportable), forming a complete “water treatment plant” or deployed as stand-alone units. The device, method, and computer process for the separation and concentration of algal biomass via ultrafiltration separate algal species from an aqueous or marine culture in a manner that minimizes the irreparable fouling of the ultrafiltration membranes and concentrates the algal biomass at low energy utilization without airlock controlled via specifically designed software for algal separation.

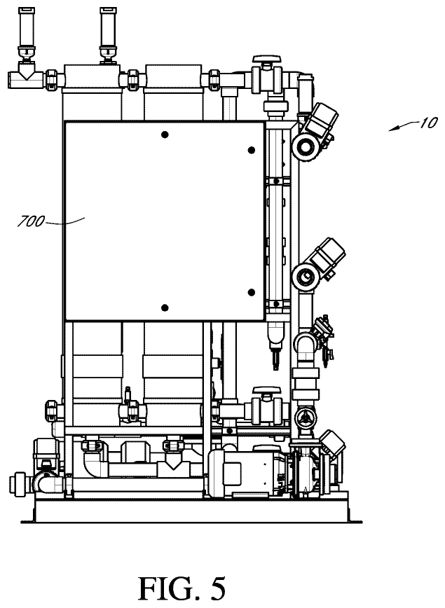

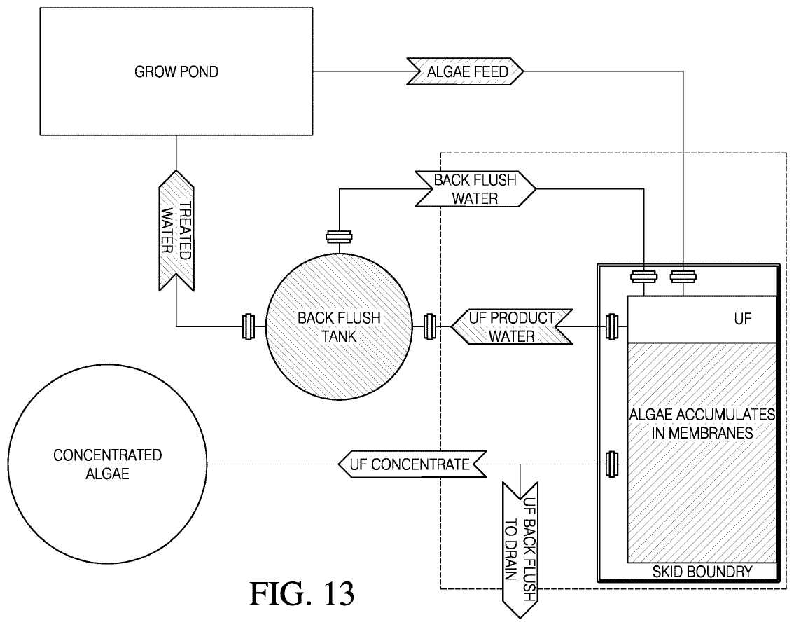

With reference to FIG. 5, device 10 includes control panel 700. Control panel 700 comprises a programmable logic controller (PLC), touchscreen interface, connections for external control, power supply, and pumps and is in communication with transmitters and sensors of the device 10. Methods for use with programmable logic controller and control panel 700 are discussed in further detail below with respect to FIG. 13.

FIG. 13 is a flow diagram of a method for concentrating and collecting algal biomass. The first step of the method comprises separating and concentrating algal biomass. An aqueous or marine culture containing algae biomass, whether directly fed or after a pretreatment step (such as gravity filtration, flocculation, air floatation, or other methods) is fed to the T-Series ultrafiltration system (device). Permeate water may be recycled to feed pond or bioreactor with valuable TDS. Algae accumulates in the pressurized collection housing.

After algae accumulates in the pressurized collection housing, air is delivered via separate air induction lines to each pressurized collection housing, creating vacuum pressure which combined with gravity rapidly collects the concentrated algae product via the device's harvesting line without causing debilitating airlock.

After collection, the device utilizes a regeneration process comprising an air scour-forward flush and air scour-back flush process which regenerates the membrane back to its original operational values.

During both the forward flush and back flush processes, air bubbles are injected into the collection housing assembly dislodging suspended solids from the hollow fiber membrane. This enables the forward and back flush process to effectively remove the solids to prevent build-up.

At a preset time or upon reaching a predetermined transmembrane pressure (TMP), the device will engage in a regeneration process. Beginning with the forward flush process, 95% of the solids are removed with the feed water without TMP constriction.

After the forward flush process has removed the majority of the solids from the collection housing assembly, ultrafiltration systems will engage in a back flush process utilizing filtrate (permeate) to back flush from the inside of the hollow fibers, hence dislodging any of the remaining solids attached to the outer surface of the fiber membrane and the flush liquid is drained from the collection housing assembly.

The device feeds the aqueous feed source at a high rate of flow in order to immediately start separating and concentrating algal biomass without the delay of filling the collection housings.

### Example 1

(PAN) Polyacrylonitrile Hollow Fiber Membranes are contained within a pressurized collection housing with three to five ports separated by flexible valves for algae separation, concentration, and collection. The device comprises one or more pressurized collection housings containing hundreds to thousands of (PAN) polyacrylonitrile hollow fiber ultrafiltration membranes with three to five ports for connecting feed lines, permeate lines, reject lines, backflush lines, harvesting lines, and air lines.

The UF membranes comprise one or more types of (PAN) polyacrylonitrile ultrafiltration membranes prepared via NIPS, NTIPS, or TIPS with variable pore structures, hydrophilicity, and support materials in different lumen sizes or lengths to create the total active surface area per collection housing.

The device may have a single collection housing comprising a single membrane or may have a combination of multiple membranes and collection housings. The UF membranes may or may not be enhanced or modified by additional chemical solutions to increase hydrophilicity. The UF membranes may or may not be enhanced or modified by additional chemical solutions to reduce fouling. The UF membranes may have asymmetric or symmetric pores ranging in size from 0.005-0.5 microns.

The pressurized collection housings containing the UF membranes have three to five ports for connecting, feed lines, permeate lines, reject lines, backflush lines, harvesting lines, and air lines with flexible valves at each port to isolate each collection housing or collection housing pair, for increased algal yield concentration and collection depending on the concentration target, feed concentration, time, and total amount of aqueous or marine culture.

The device further includes a harvest line connected to the pressurized collection housing having a flow totalizer, control valves, pressure transmitters, and spectrophotometer. The device utilizes a method for an air assisted gravity collection process by creating a vacuum pressure to rapidly collect the algae concentration via an air-assisted gravity drain detailed functions

### Example 2

A method is provided for multiple processes for the separation and concentration of algal biomass from an aqueous or marine culture in a manner that minimizes the irreparable fouling of the UF membranes and concentrates the algal product in pressurized collection housings at low energy, utilizing (PAN) Polyacrylonitrile Hollow Fiber Membranes with flexible valves in an outside-in, dead-end configuration, continuous or batch feed and membrane regeneration with air scour-forward flush/air scour-back flush. The concentration rate target and time are determined by the algal species, aqueous feed concentration of algal species, total aqueous feed, and number of membranes selected.

Algae is separated and concentrated from the aqueous solution in pressurized collection housings by feeding the aqueous feed product at low pressures ranging from 0.04-50 PSI. The operating pressures are determined by the concentration, time restrictions, and type of algae species in the aqueous or marine culture and the appropriate level of flux rate to minimize irreparable fouling of the ultrafiltration membranes.

The feed pressure is based on the concentration, time restrictions, membrane area, and type of algae species in the aqueous or marine culture. The pump feed is engaged to feed the aqueous or marine culture to the UF membrane with the rejection valve closed and permeate valve open, thus creating dead-end filtration for the algae product to be concentrated in the pressurized collection housings. The pore size determines selectivity, allowing the permeate to pass through the hollow fiber membrane from outside to inside.

The aqueous culture is delivered to the membranes via a VFD-controlled feed pump at a rate and pressure determined by the algal species (size and fouling characteristics), feed concentration of algal species, total aqueous feed, number of membranes, concentration target, and time.

The device concentrates the algal aqueous or marine culture in one or more pressurized collection housings and is collected via an air-assisted algae concentrate collection process via the harvest line. Algal biomass concentrate collection via the harvest line is determined by time, pressure or concentrate calculation by a computer system wherein the permeate valve, rejection valve, and feed valve are closed and the air-vent, harvest valve, and blower valve are opened and the blower is engaged, enabling the concentrated algal product to rapidly flush the pressurized housing by created vacuum pressure and gravity by tangential flow into a harvest line for collection.

### Example 3

After biomass concentrate collection, the device feeds the aqueous feed source at a high rate of flow until all algal concentrate housings are full, in order to immediately start separation and concentration of algal biomass without the delay of filling the collection housing.

After the air-assisted algae concentrate collection is complete, the pressurized collection housings are empty of the algal concentrate. The device begins membrane regeneration if needed. Once finished, the device begins rapid refill closing the permeate valve and opening the reject valve, and engaging the feed pump at a high rate of flow to rapidly refill all the pressurized collection housings with the aqueous feed source, reducing the downtime associated with refilling the housings.

### Example 4

A computer system may control the concentration and collection device. A computer device having steps performed that are designed for the device and methods above, the computer system allows users to make data-driven decisions specifically related to algae separation, concentration, and collection. The computer system provides a user interface via touchscreen controls and is designed to operate all of the components in the manner described above to achieve separation and concentration, as described in the examples above. The computer system also controls the components of the device including, feed and back flush pumps, and control valves to initiate processes as described above based on factors including but not limited to, feed flow, time, concentrate rate, feed concentration, concentrate target, pressure differential and TMP.

With reference to FIG. 13, a method that may be executed by a computer system for algae separation, concentration, and collection is provided. The method is designed to operate in coordination with the flexible design of the device, in batch mode, continuous mode, or a preprogrammed hybrid of the device to algal biomass product yields by collecting 98-99% of the concentrated product.

The device, method, and computer system for the separation and concentration of algal biomass via ultrafiltration separate algal species from an aqueous or marine culture in a manner that minimizes the irreparable fouling of the ultrafiltration membranes and concentrates the (variable amounts of algal concentration in the feed and total aqueous solution) algal biomass at low energy utilization without airlock controlled via specifically designed software for algal separation working in conjunction or independently. In embodiments, the computer system has a computer processor in communication with transmitters and sensors located on the device and collects and manages the feed flow, permeate flow, total algal collection, backflow flush, the inlet and outlet psi of the prefilter, the inlet-outlet psi of the membranes, the feed concentration of the algal biomass, the collected concentration of the algal biomass, and the feed temperature and feed PH. This information can be displayed and interacted with by a user at the user interface of the control panel.

### Example 5

A T-Series ultrafiltration system (device) is assembled in a manner consistent with standard skid-based or large-scale ultrafiltration plants comprised of hydrophilic modified polyacrylonitrile (PAN) hollow fiber membranes, collection housings with ports connecting the permeate line, reject line, feed line, blower line, and harvest line, and separated with flexible valves.

The device also incorporates a prefilter, feed pump, back flush pump, air blower, programmable logic controllers (PLC), control panel (and all components within), software, VFD's, automatic valves, check valves, manifolds, flow meters, pressure meters, low-pressure air valves and other components working in conjunction to separate, concentrate, and collect algal biomass from an aqueous or marine culture in the method described below.

The feed line comprises a feed pump, flow meter, prefilter housings, control valves, pressure transmitters, check valves, flexible valves, spectrophotometer, and other standard components required for proper system engineering.

The permeate line comprises a flow meter, control valve, pressure transmitters, check valves, flexible valves, back flush pump, and other standard components required for proper system engineering.

Th reject (or drain) line comprises a flow meter, control valve, and low-pressure air vent valve, flexible valves, and other standard components required for proper system engineering.

The harvest line comprises a flow totalizer, control valves, pressure transmitters, flexible valves, a spectrophotometer, and other standard components required for proper system engineering.

The blower line (air induction line) comprises a blower, control valve, check valve, flexible valves, and other standard components required for proper system engineering.

The control panel comprises a programmable logic controller (PLC) with software, touchscreen interface, connections for external control, power supply, VFD's and other components required for proper system operation.

The device can be configured into one system as an on-site installation, or as a mobile unit. In another configuration, each unit is designed and configured for easy access on a trailer as a modular unit that can be mobile (transportable), forming a complete “water treatment plant” or deployed as stand-alone units.

The method for separation, concentration, and collection of algal biomass from aqueous or marine culture comprises the following process steps all combined to equal a single “cycle”:

### Cycle

Process #1—Separation/Concentration
Process #2—Air Assisted Algae Concentrate Collection
Process #3—Membrane Regeneration
Process #4—Rapid Refill

### Process Description:

An aqueous or marine culture containing algae biomass, whether directly fed or after a pretreatment step (such as gravity filtration, flocculation, air floatation, or other methods) is fed to the T-Series ultrafiltration membrane system (device or devices).

### System Component Configuration:

The ultrafiltration system is designed with one to an infinite number of Polyacrylonitrile (PAN) hollow-fiber membranes in an outside-in, dead-end configuration, with a continuous or batch aqueous feed. The number of collection housings containing the hollow fiber membranes is determined by the amount of aqueous feed needed to be processed, the concentrate rate target, and time.

### Process Description:

Air is delivered via separate air induction lines to each pressurized collection housing, creating vacuum pressure which, combined with gravity, rapidly collects the concentrated algae product via the device's harvesting without causing debilitating airlock.

### Process Description:

The device utilizes a regeneration process comprising an air scour-forward flush and air scour-back flush process which regenerates the membrane back to its original operational values.

### Air Scour:

During the back flush, forward flush, and concentrate collection process, air bubbles are injected into the collection housing, dislodging the algal cake layer from the hollow fiber (PAN) membrane, thus enabling the forward flush and back flush process to effectively remove the solids from the membrane to regenerate the membrane to its original operational values.

### Forward Flush Process:

The device feeds the aqueous feed source at a flow rate and pressure determined by the calculation described in “Process #1” in a tangential flow across the membrane, removing 95% of the solids in the collection housing without the transmembrane pressure (TMP) restrictions.

### Back Flush Process:

The device feeds permeate at a flow rate and pressure determined by “Process #1” in an inside-out flow path, through the (PAN) membrane, dislodging algae from the pores and regenerating the membrane surface.

In summary, a device is described. The device includes a filter membrane and a collection housing assembly for housing the filter membrane in a dead-end configuration and collecting algal biomass. The device further includes an inlet for feeding influent liquid into the collection housing assembly and through the filter membrane in a dead-end configuration, where the filter membrane separates algal biomass from the influent liquid, and the algal biomass is collected at the bottom of the collection housing assembly. The device includes a first outlet for the filtered influent liquid to flow away from the filter membrane and out of the collection housing assembly and a valve that is open during feeding influent liquid and closed during the removal of algal biomass. The device has an air blower for removing algal biomass from the collection housing assembly when the inlet stops feeding influent liquid, and a second outlet for removing the algal biomass from the collection housing assembly and the device without adding additional water after the inlet stops feeding influent liquid. In some configurations, the inlet serves as the outlet for removing the biomass when the valve is in the closed position. The influent liquid is algae grow pond liquid and the filter membrane is a hydrophilic membrane. The collection housing assembly is pressurized, and the pressure is decreased to remove the algal biomass from the collection housing assembly.

In a second configuration, a device includes a filter membrane and a collection housing assembly for housing the filter membrane in a dead-end configuration and collecting biomass. The device further includes an inlet for feeding influent liquid through the filter membrane in the dead-end configuration, where the filter membrane separates biomass from the influent liquid and the biomass is collected in the collection housing assembly. The device includes an air blower for removing biomass from the collection housing assembly when the inlet stops feeding influent liquid and an outlet for removing the biomass from the collection housing assembly. In some configurations, the device includes a valve in an open position to feed influent liquid through the filter membrane. The valve in a closed position stops the inlet from feeding influent liquid to the filter membrane. The inlet can serve as the outlet for removing the biomass when the valve is in the closed position. The outlet removes the biomass solids without additional water being added to the collection housing assembly when the valve is in the closed position.

The device further includes a second outlet for removing filtered liquid from the collection housing assembly. The influent liquid may be algae grow pond liquid and the biomass may be algae. The filter membrane may be a hydrophilic membrane. The collection housing assembly may be pressurized and pressure in the collection housing assembly may be decreased to remove the biomass from the collection housing assembly.

A method is provided for flowing algae grow pond liquid through a filter membrane, where a collection housing assembly maintains the filter membrane in a dead-end configuration. Algal biomass is filtered from the algae grow pond liquid. The algal biomass is collected in the collection housing assembly.

In response to stopping the flow of algae grow pond liquid, the pressure in the collection housing assembly is decreased and the algal biomass is removed from the collection housing assembly through an outlet. In some configurations, the algal biomass is collected in a bottom of the collection housing assembly, where the algal biomass is not in contact with the filter membrane. The algal biomass may be removed from the collection housing assembly without additional water being added to the collection housing assembly.

The collection housing assembly may be pressurized, and the pressure is decreased in the collection housing assembly when removing the algal biomass from the collection housing assembly.

1. A device comprising:
a filter membrane;
a collection housing assembly housing the filter membrane in a dead-end configuration and collecting algal biomass;
an inlet feeding influent liquid into the collection housing assembly and through the filter membrane in a dead-end configuration, wherein the filter membrane separates algal biomass from the influent liquid and the algal biomass is collected at a bottom of the collection housing assembly;
a first outlet for filtered influent liquid to flow away from the filter membrane and out of the collection housing assembly;
a valve that is open during feeding of the influent liquid and closed during removal of the algal biomass;
an air blower removing the algal biomass from the collection housing assembly when the inlet stops feeding influent liquid; and
a second outlet removing the algal biomass from the collection housing assembly and the device without adding additional water after the inlet stops feeding the influent liquid.

2. The device of claim 1, after the inlet serves as the second outlet removing the biomass when the valve is in a closed position.

3. The device of claim 1, wherein the influent liquid is algae grow pond liquid.

4. The device of claim 1, wherein the filter membrane is a hydrophilic membrane.

5. The device of claim 1, wherein the collection housing assembly is pressurized, and pressure is decreased to remove the algal biomass from the collection housing assembly.

6. The device of claim 1, wherein substantially all of the influent liquid flows through the filter membrane.

7. A device comprising:
a filter membrane;
a collection housing assembly housing the filter membrane in a dead-end configuration and collecting biomass;
an inlet feeding influent liquid through the filter membrane in the dead-end configuration, wherein the filter membrane separates the biomass from the influent liquid and the biomass is collected in the collection housing assembly;
an air blower removing biomass from the collection housing assembly when the inlet stops feeding the influent liquid; and
an outlet removing the biomass from the collection housing assembly.

8. The device of claim 7, further comprising:
a valve in an open position to feed the influent liquid through the filter membrane.

9. The device of claim 8, wherein the valve in a closed position stops the inlet from feeding the influent liquid through the filter membrane.

10. The device of claim 9, wherein the inlet serves as the outlet for removing the biomass when the valve is in the closed position.

11. The device of claim 10, wherein the outlet removes the biomass without additional water being added to the collection housing assembly when the valve is in the closed position.

12. The device of claim 7, further comprising:
a second outlet removing filtered liquid from the collection housing assembly.

13. The device of claim 7, wherein the influent liquid is algae grow pond liquid.

14. The device of claim 13, wherein the biomass is algae.

15. The device of claim 7, wherein the filter membrane is a hydrophilic membrane.

16. The device of claim 7, wherein pressure in the collection housing assembly is decreased to remove the biomass from the collection housing assembly.

17. A method comprising:
flowing algae grow pond liquid through a filter membrane, wherein a collection housing assembly maintains the filter membrane in a dead-end configuration;
filtering algal biomass from the algae grow pond liquid;
collecting the algal biomass in the collection housing assembly;
stopping flow of algae grow pond liquid through the filter membrane;
in response to stopping flow of algae grow pond liquid, decreasing pressure in the collection housing assembly; and
removing the algal biomass from the collection housing assembly through an outlet.

18. The method of claim 17, further comprising:
collecting the algal biomass in a bottom of the collection housing assembly, wherein the algal biomass is not in contact with the filter membrane.

19. The method of claim 18, further comprising:
removing the algal biomass without additional water being added to the collection housing assembly.

20. The method of claim 19, further comprising:
decreasing pressure in the collection housing assembly when removing the algal biomass from the collection housing assembly.
